(19) United States
(12) Patent Application Publication
(10) Pub. No.: US 2010/0249637 A1
(43) Pub. Date: Sep. 30, 2010

(54) SYSTEMS, DEVICES, AND METHODS FOR TREATING RESTLESS LEG SYNDROME AND PERIODIC LIMB MOVEMENT DISORDER

(75) Inventors: Timothy J. Walter, Upper Arlington, OH (US); Uma Marar, Blacklick, OH (US)

Correspondence Address: MUETING, RAASCH & GEBHARDT, P.A., P.O. BOX 581336, MINNEAPOLIS, MN, 55458-1336, US

(73) Assignee: Lotus Magnus, LLC, Grove City, OH (US)

(21) Appl. No.: 12/438,267

(22) PCT Filed: May 8, 2008
(86) PCT No.: PCT/US08/63006

§ 371 Date: Feb. 20, 2009

(51) Int. Cl. A61B 5/04; A61H 1/00

(52) U.S. Cl. 600/544; 601/46

(57) ABSTRACT

Systems, devices, and methods for treating restless leg syndrome and periodic limb movement disorder that may include sleeve apparatus and vibration apparatus to stimulate the limb(s) of a subject.

The present invention relates generally to systems, devices, and methods for treating restless leg syndrome and periodic limb movement disorder.

Restless Leg Syndrome (RLS) is a medical condition that may cause a subject to have unpleasant sensations in their legs. RLS-like symptoms may affect portions of the anatomy other than legs, such as arms, trunk, neck, shoulders, arms, face, etc. The unpleasant sensations are particularly prevalent when the subject is at rest. Further, the longer a subject is at rest, the greater the chance the sensations will occur and the more severe the sensations may be. The symptoms are often the most problematic when the subject is trying to fall asleep, but may also occur when the subject is sitting (e.g., sitting for longer periods of time in an automobile, airplane, office, theater, classroom, etc.) or when the subject may awaken from asleep.

RLS is typically characterized by sensations of deep-seated tingling, burning, aching, creeping, itching, pulling, tugging, gnawing, etc. These sensations contribute to the subject's compulsion to move their affected limbs. Forcing the limbs to remain still may cause the subject to incur additional discomfort and/or involuntary jerking of the limbs.

A substantial number of subjects who have RLS also have periodic limb movement disorder (PLMD; also known as nocturnal myoclonus). PLMD may be characterized by repeated involuntary leg twitching or jerking of the limbs during sleep that may continue for about 0.5 seconds to about 5 seconds and may occur at intervals of about seconds 10 to about 60 seconds. RLS/PLMD can cause a subject to experience fragmented, disrupted sleep.

RLS/PLMD is often treated with minor changes in diet (e.g., minimizing caffeine, alcohol, tobacco, nicotine, etc.), pharmacologic treatments (e.g., benzodiazepines, dopamine agonists, carbidopa-levodopa, anti-convulsants, opioids, etc.), vitamins (e.g., iron, folate, magnesium, etc.), and/or physical treatments (e.g., taking a hot bath, massaging the legs, using a heating pad or ice pack). Although many subjects find some relief with such measures, rarely do these efforts eliminate symptoms.

One method for treating RLS has been described in, e.g., U.S. Patent Application Pub. No. 2005/0026912 A1, published 3 Feb. 2005 to Morgenlander and entitled “Method of Treating Restless Leg Syndrome” that describes a method to treat RLS by utilizing a fluid-filled sleeve to apply pressure to the limbs of a subject over periods of time.

### SUMMARY OF THE INVENTION

In one aspect, the present invention provides a system for treating restless leg syndrome and periodic limb movement disorder. The system includes sleeve apparatus to surround a portion of a subject; vibration apparatus coupled to the sleeve to vibrate the sleeve; and motion sensing apparatus to detect motion of the portion of the subject.

In another aspect, the present invention provides a method for treating restless leg syndrome and periodic limb movement disorder. The method includes providing a treatment system. The treatment system includes a sleeve to surround a portion of a subject and vibration apparatus coupled to the sleeve to vibrate the sleeve. The method further includes locating the sleeve on the leg of the patient, and vibrating the sleeve with the vibration apparatus.

The above summary is not intended to describe each embodiment or every implementation of the present invention. Rather, a more complete understanding of the invention will become apparent and appreciated by reference to the following Detailed Description of Exemplary Embodiments and claims in view of the accompanying figures of the drawing.

### BRIEF DESCRIPTION OF THE DRAWING

FIG. 1A is an illustrative view of one exemplary embodiment of a RLS/PLMD treatment system according to the present invention.

FIG. 1B is an illustrative view of another exemplary embodiment of a RLS/PLMD treatment system according to the present invention.

FIG. 1C is an illustrative view of another exemplary embodiment of a RLS/PLMD treatment system according to the present invention.

FIG. 1D is a diagrammatic representation of another exemplary embodiment of a RLS/PLMD treatment system according to the present invention.

FIG. 2 is a diagrammatic representation of another exemplary embodiment of a RLS/PLMD treatment system according to the present invention.

FIG. 3 is a diagrammatic representation of another exemplary embodiment of a RLS/PLMD treatment system according to the present invention.

FIG. 4 is a flow chart of one exemplary method of treating RLS/PLMD according to the present invention.

FIG. 5 is a flow chart of another exemplary method of treating RLS/PLMD according to the present invention.

### DETAILED DESCRIPTION OF EXEMPLARY EMBODIMENTS

In the following detailed description of illustrative embodiments of the invention, reference is made to the accompanying figures of the drawing which form a part hereof, and in which are shown, by way of illustration, specific embodiments in which the invention may be practiced. It is to be understood that other embodiments may be utilized and structural changes may be made without departing from the scope of the present invention. Unless stated otherwise herein, the figures of the drawing are rendered primarily for clarity and thus may not be drawn to scale.

As used herein, “a,” “an” “the,” “at least one,” and “one or more” are used interchangeably. The term “and/or” (if used) means one or all of the listed elements or a combination of any two or more of the listed elements.

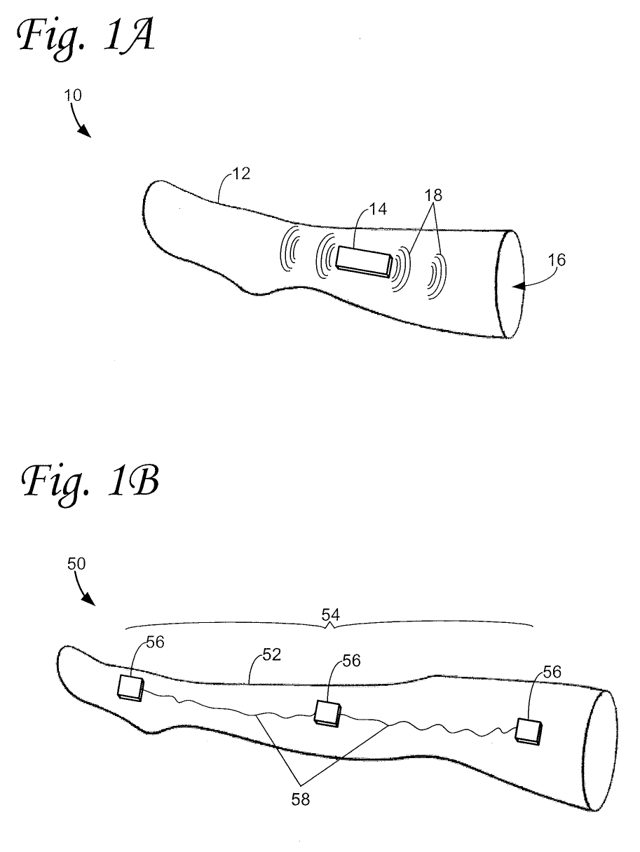

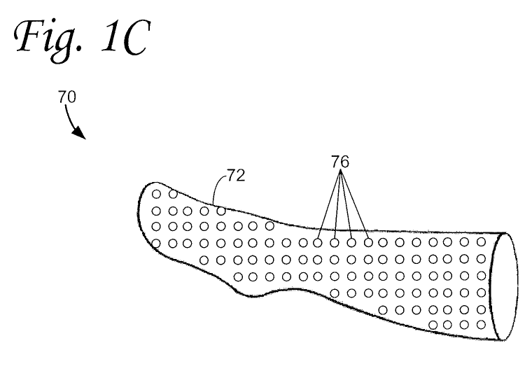

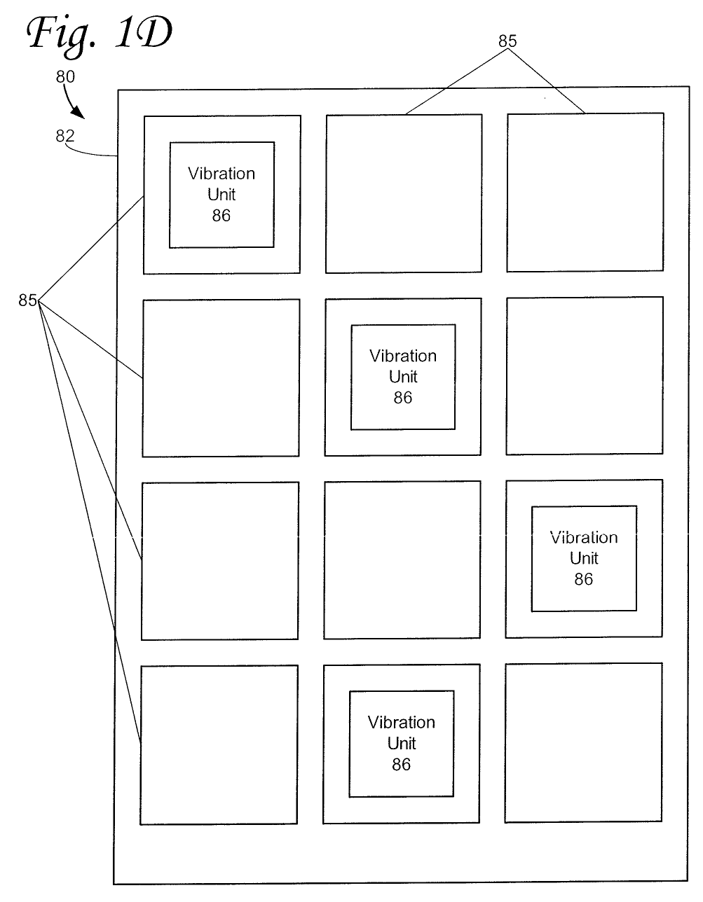

FIG. 1A depicts a RLS/PLMD treatment system 10 according to the present invention. The RLS/PLMD treatment system 10 may include a sleeve 12 and vibration apparatus 14.

The sleeve 12 may be sized to fit around a portion of a subject's anatomy such as, e.g., the leg. In the embodiment depicted in FIG. 1A, the sleeve 12 may surround a subject's foot and calf. Further, although the sleeve 12 shown in FIG. 1A covers the subject's foot, the sleeve 12 may only extend from a subject's ankle to a subject's knee leaving the foot uncovered (e.g., the sleeve may be open on either end). In one embodiment, the sleeve may have an adjustable longitudinal length such that a subject can adjust how much of his/her leg may be covered by the sleeve.

Although the description provided herein describes the sleeve of the RLS/PLMD treatment system according to the present invention as surround a subject's leg, the sleeve may be designed to surround or partially-surround any portion of a subject's body (e.g., the sleeve may be designed to surround a subject's arm from wrist to armpit, a subject's trunk, etc.).

Further, although the sleeve 12 may be tubular as shown, the sleeve may not completely surround a subject's leg (e.g., the sleeve may be U-shaped like a sling). In one embodiment, the sleeve may a sheet of material that may be wrapped around the limb of the subject and attached with a fastener (e.g., a strap, buckles, mechanical interlocking fasteners, hook and loop closures, adhesives, etc.). The sleeve 12 includes an opening 16, within which a subject may insert their leg into the sleeve 12. In other embodiments, the sleeve 12 may have other openings (e.g., openings for a subject's toe, vents, etc.).

The sleeve 12 may be formed of any skin-compatible material such as, e.g., cotton, wool, nylon, polymer, neoprene, etc. In other embodiments, the sleeve 12 may be formed of multiple different types of materials including compliant, semi-compliant, non-compliant, elastic, non-elastic materials. The sleeve may also include structures to retain the sleeve on a subject's leg. For example, the sleeve may include an elastic band around the opening 16 to keep the sleeve 12 “pulled-up.” Further, the sleeve may include a zipper that, e.g., may run the length of the sleeve to assist a subject in placing the sleeve on his/her leg.

The RLS/PLMD treatment system 10 may further include vibration apparatus 14. Vibration apparatus 14 according to the present invention may be any apparatus that is capable of emitting vibrations to vibrate the sleeve 12. Such vibrations may be in the frequency range of about 1 hertz or more, about 50 hertz or more, about 65 hertz or more, about 100 hertz or more, about 150 hertz or more, about 200 hertz or less, about 500 hertz or less, etc. In the embodiment depicted in FIG. 1A, the vibration apparatus 14 is a single rectangular unit that is attached to the sleeve 12. Vibrations 18 are depicted in FIG. 1A to show how vibrations may travel from the vibration apparatus 14 throughout the sleeve 12.

The vibration apparatus 14 may include any item that may be necessary to provide vibrations according to the present invention. For example, the vibration apparatus 14 may include power sources, controllers, microprocessors, digital signal processors, electric motors, mechanical motors, rotors, shafts, piezoelectric vibration devices, switches, memory, dials, timers, sound apparatus, visual indicators, input/output ports, power inputs, digital displays, and any other device that may be useful in providing vibrations to the sleeve 12. Further, the vibration apparatus 14 may be embedded or woven-into the sleeve 12. Although the vibration apparatus 14 as depicted in FIG. 1A extends outwardly from the sleeve 12 (i.e., box-like), vibration apparatus may be any size and/or shape. For example, the vibration apparatus may be small and flat so as to be unobtrusive to the wearer. Also, for example, the vibration apparatus may not be viewable to the wearer; instead, the vibration apparatus may be wholly embedded between layers of the sleeve.

The materials that form the sleeve 12 are preferably capable of transmitting vibrations throughout the sleeve 12. However, the sleeve 12 may also include vibration transmitting structures, such as semi-compliant strands, structures, etc. that extend around or through the sleeve 12 to transmit vibrations away from the vibration apparatus 14. The vibration transmitting structures may, e.g., extend throughout the sleeve in a “spider web-like” or “fishing net-like” manner.

FIG. 1B depicts another RLS/PLMD treatment system 50 according to the present invention. The RLS/PLMD treatment system 50 may include a sleeve 52 and vibration apparatus 54. The RLS/PLMD treatment system 50 may be different from the RLS/PLMD treatment system 10 depicted in FIG. 1A such that the system 50 includes a longer sleeve 52 and the vibration apparatus 54 includes multiple vibration units 56.

The sleeve 52 may extend from a subject's foot to about mid-thigh (instead of extending from the subject's foot to about calf as sleeve 12). Further, sleeve 52 has vibration apparatus 54 that includes multiple vibration units 56. Each vibration unit 56 may be substantially similar to the vibration apparatus 14 as described herein with reference to FIG. 1A. Further, for example, the vibration units may be attachable to any location on the sleeve by, e.g., hook and loop closures, adhesives, etc.

The vibration units 56 may optionally be electrically coupled to each other with a wire 58 that may be embedded or woven-into the sleeve 52. In other embodiments, the vibration apparatus 54 may include multiple vibration units 56 that are not coupled, or only a few multiple vibration units that are coupled. The vibration units 56 may emit vibrations in unison, independently, intermittently, in a selected order, in a staggered order, etc. and may share electrical power with one another. In other embodiments, one of the vibration units 56 may include a power source and the other vibration units may receive power from that vibration unit. Further, only one of the vibration units 56 may include a controller device, etc. that controls the other vibration units 56. For example, one vibration unit may include a dial that may activate the units and may adjust the frequency at which they vibrate.

FIG. 1C depicts another RLS/PLMD treatment system 70 according to the present invention. The RLS/PLMD treatment system 70 may include a sleeve 72 including an array of vibration units 76. The vibration units 76 as depicted are button shaped and arranged in an array so as to cover the entire sleeve 72. Each vibration unit 76 may be connected to one another by wires (not shown) that may be embedded within the sleeve.

Although the vibration units 76 are shown in a uniform, rectilinear array, the vibration units may be arranged and/or located anywhere on the sleeve. Each pair of adjacent vibration units may be spaced apart by any selected distance, e.g., about 10 millimeters or more, about 50 millimeters or more, about 75 millimeters or less, 150 millimeters or less, etc. The vibration units may be, in some embodiments, spaced apart by distances that are related to the sensitivity of the portion of the subject's anatomy (e.g., using two-point discrimination) over which the vibration units are located.

The array of vibration units may emit vibrations in unison, independently, intermittently, in a selected order, in a staggered order, etc. The vibration units may vibrate such that the vibrations sweep across the sleeve from the distal end (e.g., the end proximate the subject's foot) to the proximal end (e.g., the end proximate the opening) or vice versa. In other embodiments, the vibration units proximate the distal end of the sleeve may vibrate with greater intensity than the vibration units proximate the proximal end of the sleeve since, in some instances, subjects have more intense sensations proximate the distal end of their limb.

Although the RLS/PLMD treatment systems 10, 50, 70 in FIGS. 1A-1C are depicted as including a single sleeve, RLS/PLMD treatment systems according to the present invention may include multiple sleeves for contacting multiple different limbs of a subject (e.g., legs, arms, neck, trunk, face, or any combination). In these embodiments, the vibration apparatus may include multiple units that may be coupled to each other (e.g., wirelessly coupled). In other embodiments, the RLS/PLMD treatment system may be in the form of one or more adhesive patches that may be attached to any portions of a subject's anatomy.

FIG. 1D is a diagrammatic representation of another RLS/PLMD treatment system 80 according to the present invention. The RLS/PLMD treatment system 80 may include a sleeve 82 including multiple pockets 85 and multiple vibration units 86. Although FIG. 1D is a diagrammatic representation, the sleeve 82 may be substantially similar to the sleeves 12, 52, and 72 as described herein. The pockets 85 may be located anywhere on the sleeve 82 and may be configured to receive a vibration unit 86 such that a subject may remove a vibration unit 86 from a pocket 85 and place the vibration unit 86 into a different pocket 85. It may be preferred that the number of pockets 85 be greater than the number of vibration units 86 such that one or more of the pockets 85 do not contain a vibration unit 86. Further, pockets 85 may be located on the inside or outside of the sleeve 82 and may include a closure mechanism (e.g., a hook and loop closure) for securing the vibration unit 86 within pocket. In this embodiment, a subject may move the vibration units 86 to locations of the sleeve 82 so as to, e.g., not interfere with his/her sleeping position, apply vibrational stimulation to selected portions of the subject's anatomy, etc.

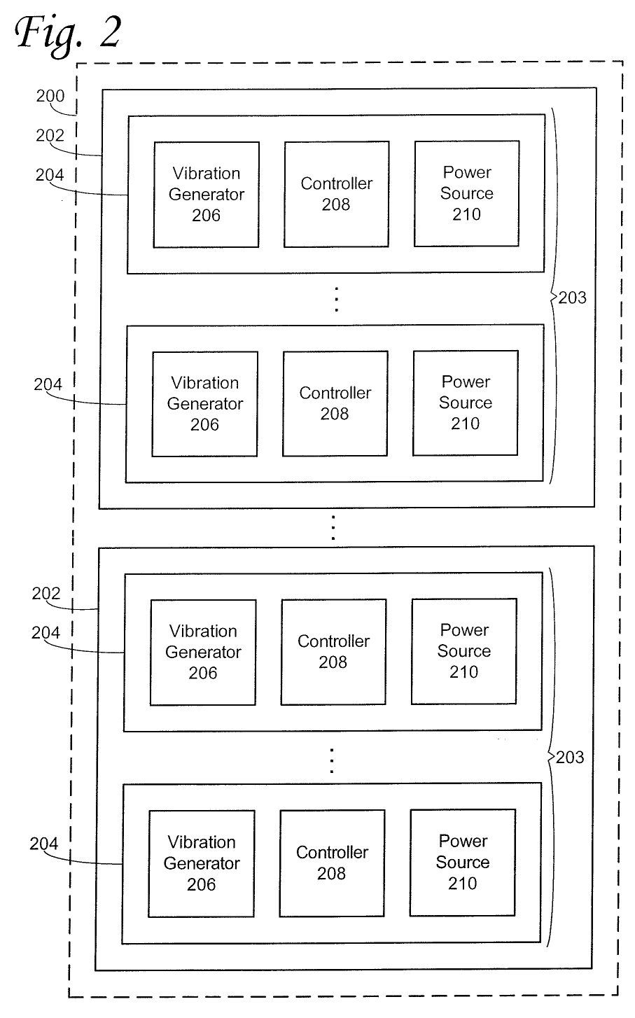

FIG. 2 is a diagrammatic representation of another exemplary embodiment of a RLS/PLMD treatment system 200 according to the present invention. The RLS/PLMD treatment system 200 may include two or more sleeves 202 (as noted by the ellipsis between the sleeves 202). Each sleeve 202 may include vibration apparatus 203 that may include two or more vibration units 204 (as noted by the ellipses between the vibration units 204).

Each vibration unit 204 may include a vibration generator 206, a controller 208, and a power source 210. The vibration generator 206 may be a DC motor capable of rotating a shaft and a weight carried off-center on the shaft (e.g., an eccentric weight), a piezoelectric vibration generator, etc.

The controller 208 may be any microprocessor, microcontroller, digital signal processor, analog circuit, digital circuit, etc. that is capable of controlling the vibration units 204. The controller 208 may be electrically coupled (e.g., with a wire, via wireless communication, etc.) to the vibration generators 206 and/or the power sources 210. The controller 208 may include memory that may be programmable to control the vibrational speed, duration, amplitude, timing, staggering, etc. of the vibration units 204. The controller 208 may include inputs (e.g., switches, buttons, dials, etc.) that may be used by the subject to control various attributes of the system 200 and/or program the controller 208. For instance, the controller 208 may be able to be pre-programmed by a subject to deliver a selected program of vibrations for an extended period of time. For example, a subject may program the controller 208 such that the sleeves 202 vibrate at 100 hertz for 30 minutes, then 65 hertz for 30 minutes, and then turn “off.” Further, for example, a subject may program the controller 208 such that the sleeves 202 cycle between vibrating at 85 hertz for 15 seconds and not vibrating for 45 seconds. Many other variations are possible.

The controller 208 may include an interface (e.g., serial data connection, parallel data connection, Advanced Technology Attachment (ATA), Small Computer System Interface (SCSI), Serial Advanced Technology Attachment (SATA), Universal Serial Bus, IEEE 1394, etc.) that may be couplable to a personal computer and/or memory device. The controller may be programmed through such interface using, e.g., a personal computer. In other embodiments, memory devices may be available that are programmed with at least one specific cycle or pattern.

The controller may include a power switch (although not depicted). The power switch may be any kind of two or more position switch. The power switch may have two positions: “on” and “off.” When the power switch is in the “on” position, the vibration apparatus may emit vibrations. When the power switch is in the “off” position, the vibration apparatus may be dormant, i.e., not vibrating. The power switch may have more than two positions for different modes of operation of the sleeve. For example, the power switch may have a position for an “intermittent” mode in which vibrations may be emitted intermittently (e.g., a cycle of 60 seconds of vibrations followed by 3 minutes of rest)

The vibration unit 204 may further include an indicator. The indicator may be a single LED. In other embodiments, the indicator light may consist of one or more LEDs, OLEDs, LCDs, etc. The indicator may indicate to the user the mode or state of the vibration unit 204 and/or system 200. For example, if the indicator is “on,” it may be indicating that the power source of the vibration apparatus is fully charged. Also, for example, the indicator light may “blink” to indicate the power source of the vibration apparatus is low on power.

The vibration apparatus described herein may include any analog and/or digital electronic components and/or circuits to facilitate the controlling, programming, vibrating, etc. of the RLS/PLMD treatment system 200. For example, the vibration apparatus may include capacitive electronic components (e.g., capacitors) arranged in a circuit to oscillate the electrical current at a selected frequency to create vibrations with the vibration-emitting devices (e.g., motors).

Although the separate components (i.e., vibration generator 206, controller 208, power source 210) of each vibration unit 204 are shown in FIG. 2 as being separate, such components may be completely integrated or partially integrated into a single unit. For example, such components may reside one single microelectronic chip or may be located on the same circuit board.

The power source 210 may be any electrical power source that is capable of providing power to the vibration apparatus (e.g., a battery, a fuel cell, etc.). The power source 210 may be removable or non-removable, rechargeable or non-rechargeable, etc. In other embodiments, the power source 210 may be a power input that may be electrically coupled to an external power source (e.g., a wall socket). The power source 210 may be electrically coupled to the vibration generator 206, the controller 208, and/or any other device of the sleeve 202 that may need power.

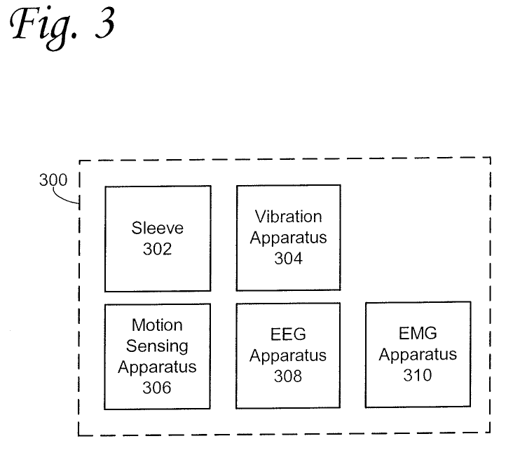

FIG. 3 is a diagrammatic representation of another exemplary embodiment of a RLS/PLMD treatment system 300 according to the present invention. The RLS/PLMD treatment system 300 may include a sleeve 302, vibration apparatus 304, motion sensing apparatus 306, electroencephalography apparatus 308, and electromyography apparatus 310. In other embodiments, a treatment system according to the present invention may include a sleeve, vibration apparatus, and any one or more of motion sensing apparatus, EEG apparatus, and EMG apparatus (e.g., a sleeve, vibration apparatus, motion sensing apparatus and EMG apparatus). The sleeve 302 and the vibration apparatus 304 may be similar to the sleeve(s) 204 and vibration apparatus 203 described herein in system 200.

Each of the vibration apparatus 304, motion sensing apparatus 306, electroencephalography apparatus 308, and electromyography apparatus 310 may be operatively electrically coupled to each other such that they may be operated in conjunction with each other for use with, e.g., the methods described herein according to the present invention. In some embodiments, the motion sensing apparatus may not be attached to the sleeve but may be provided as separate and discrete components that are operably coupled to the remainder of the system.

The motion sensing apparatus 306 may be any apparatus capable of detecting the movement of a subject. For example, the motion sensing apparatus 306 may include one or more accelerometers, mercury switches, etc. that are attached to the sleeve (e.g., sleeve 12) to detect if the legs of the subject are moving. Further, the motion sensing apparatus 306 may include multiple motion sensing devices located throughout the sleeve so that the motion of different parts of the subject's legs may be monitored. For instance, the motion sensing apparatus 306 may have a motion sensor near a subject's ankle and another motion sensor near a subject's mid-thigh to detect and/or distinguish between motions above and below the subject's knee.

The electroencephalography apparatus 308 may be any apparatus that is capable of monitoring neural activity of a subject. Electroencephalography (EEG) records the neural activity of electrical potential across cell membranes, which are detected through the cerebral cortex and recorded by a plurality of electrodes. The changes in electrical potential in the cortex contain rhythmical activity, which typically occur at frequencies of about 0.5 to about 70 cycles per second (hertz).

The EEG apparatus 308 may include one or more electrodes located proximate one or more selected portions of the subject's brain (e.g., precentral gyrus, postcentral gyms, frontal lobes, cerebellum, etc.) and may monitor the neural activity of the subject's brain to determine if the subject is moving his/her legs or other anatomical feature. For example, the EEG apparatus 308 may monitor the brain's Mu rhythm. Further, the EEG apparatus 308 may monitor the neural activity of the subject's brain to determine if the subject is about to move his/her legs. In this case, the system and/or method according to the present invention may preemptively vibrationally stimulate the subject's leg before the subject consciously moves his/her legs and/or before the subject becomes aware of the sensations to move his/her legs.

The electromyography apparatus 310 may be any apparatus that is capable of monitoring the muscular activity of a selected portion of a subject. Electromyography (EMG) records the muscular activity of electrical potential across muscular membranes, which range between about 50 microvolts to about 30 millivolts (depending on the muscle under observation). Typical repetition rate of muscle unit firing is about 7 hertz to about 200 hertz, depending on the size of the muscle, the type of muscle, etc. EMG signals may be recorded within a muscle (i.e., intramuscular EMG) or on the surface a subject's skin outside of a muscle.

The EMG apparatus 310 may include one or more electrodes located proximate one or more selected portions of the subject's body (e.g., the subject's legs) and may monitor the muscular activity of the subject's legs, etc. to determine if the subject is moving his/her legs. Further, the EMG apparatus 310 may monitor the muscular activity of the subject's legs, etc. to determine if the subject is about to move his/her leg(s). In this case, the system and/or method according to the present invention may preemptively vibrationally stimulate the subject's leg before the subject consciously moves his/her leg due to RLS/PLMD and/or before the subject becomes aware of the sensations to move his/her leg.

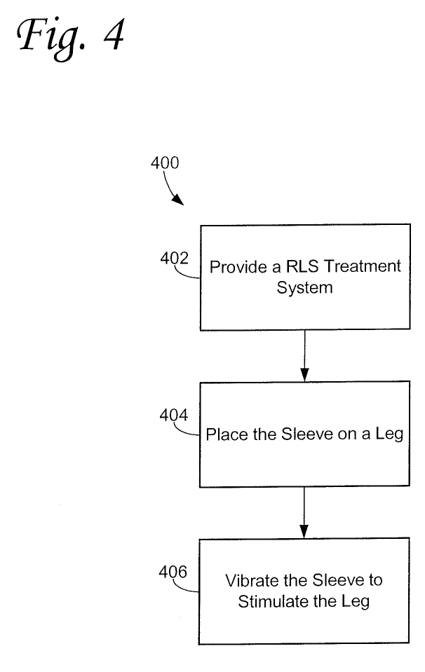

FIG. 4 depicts a flow chart of one exemplary method of treating RLS/PLMD 400 according to the present invention. The method 400 may include providing a RLS/PLMD treatment system 402 (e.g., the RLS/PLMD treatment systems 10, 50, 70 described herein with reference to FIGS. 1A-1C), locating a sleeve of the system on a leg of a subject 404, and vibrating the sleeve to stimulate the leg of the subject 406.

A subject may locate the sleeve of the system on their leg by sliding their foot into an opening (e.g., opening 16) of the sleeve to the opposite end of the sleeve (like, e.g., putting on a sock). In other embodiments, a subject may wrap the sleeve around their leg and secure it with, e.g., a strap. After placing the sleeve on their leg, a subject may activate the sleeve to vibrate, thereby stimulating their leg.

The method 400 may include vibrating the sleeve at one or more selected frequencies. In one embodiment, the method may vibrate at a first frequency and then a second frequency, wherein the first frequency is greater than the second frequency. The method may vibrate in the frequency range of about 1 hertz or more, about 50 hertz or more, about 200 hertz or less, about 500 hertz or less, etc. In other embodiments, the method may gradually and/or periodically shift the vibrations between two or more different frequencies.

The method 400 may include intermittent vibrations. For example, the RLS/PLMD treatment system may cycle between, e.g., 15 seconds of vibration and 45 seconds of rest. In other embodiments, the method may vibrate intermittently between various frequencies. For example, the RLS/PLMD treatment system may cycle between, e.g., 2 minutes of 100 hertz vibrations and 5 minutes of 50 hertz vibrations. Still further, the system may cycle between two or more states, e.g., 1 minute of 120 hertz vibrations, 5 minutes of 80 hertz vibrations, and 30 minutes of rest.

The method may include vibrating for a fixed period of time, e.g., 30 minutes. The RLS/PLMD treatment system may include a dial or any other input device with which a subject may set the length of the fixed period of time of vibration. Further, the treatment system may further include a “snooze” button that would extend the vibrations for a pre-selected period of time, e.g., 5 minutes beyond the fixed period of time. If a subject has not fallen asleep after the fixed period of time has expired, the subject may depress the “snooze” button to continue vibrations for, e.g., five more minutes.

The method may further include staggering the vibrations over multiple vibration units. For example, a vibration unit proximate the subject's foot may vibrate for 45 seconds while the other vibration units lie dormant, followed by the a vibration unit proximate the subject's calf vibrating for 45 seconds while the other vibration units lie dormant, etc. Further, the method may further include vibrating different portions of subject's leg at different frequencies (e.g., the calf at 100 hertz and the thigh at 50 hertz). In other embodiments, the method may include gradually and/or periodically shifting the vibrations between two or more different frequencies and/or two or more different portions of a subject's limb.

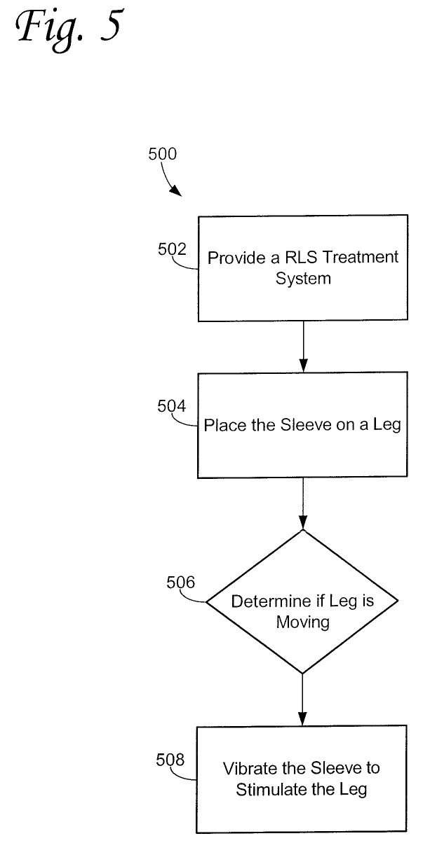

FIG. 5 depicts a flow chart of one exemplary method of treating RLS/PLMD according to the present invention. The method 500 may include providing a RLS/PLMD treatment system 502, locating a sleeve of the system on a leg of a subject 504, and vibrating the sleeve to stimulate the leg of the subject 508. Steps 502, 504 and 508 may similar step 402, 404, and 406 of the method 400 described herein.

However, method 500 further includes optionally determining if the leg of the subject is moving 506 before vibrating the sleeve to stimulate the leg 508. This determination step 506 may utilize motion sensing apparatus (e.g., motion sensing apparatus 506) that is coupled to the sleeve (e.g., sleeve 12) to detect if the leg of the subject is moving. The motion sensing apparatus may include multiple motion sensing devices located throughout the sleeve so that the motion of different parts of the subject's leg or legs may be monitored. In other embodiments, the determination step 506 may utilize the EEG apparatus 508 and/or EMG apparatus 510 to determine if the leg of the subject is moving or is about to move.

If the subject's leg is determined to be moving or is about to move, the determination step 506 may then allow the sleeve to vibrate to stimulate the leg 508. As described herein, vibrating the sleeve to stimulate the leg may occur for a selected period of time, intermittently, etc. Such a determination step 506 may be useful, e.g., when a subject is undergoing movements during sleep (e.g., subjects with PLMD) and may not be able to activate the sleeve using a switch, etc.

The complete disclosure of the patents, patent documents, and publications cited in the Background, the Detailed Description of Exemplary Embodiments, and elsewhere herein are incorporated by reference in their entirety as if each were individually incorporated.

Illustrative embodiments of this invention are discussed and reference has been made to possible variations within the scope of this invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A system for treating restless leg syndrome and periodic limb movement disorder, the system comprising:
sleeve apparatus to surround a portion of a subject;
vibration apparatus coupled to the sleeve to vibrate the sleeve; and
motion sensing apparatus to detect motion of the portion of the subject.

2. The system of claim 1, wherein the motion sensing apparatus comprises EMG apparatus to detect motion of the portion of the subject.

3. The system of claim 1, wherein the motion sensing apparatus comprises EEG apparatus to detect motion of the portion of the subject.

4. The system of claim 1, wherein the motion sensing apparatus comprises an accelerometer.

5. The system of claim 1, wherein the vibration apparatus comprises two or more vibration units spaced apart from each other.

6. The system of claim 5, wherein each of the two or more vibration units are attachable to the sleeve.

7. The system of claim 5, wherein the sleeve comprises a plurality of pockets, and wherein each of the two or more vibration units is locatable within one of the plurality of pockets.

8. The system of claim 7, wherein the number of pockets is greater than the number of vibration units.

9. A method for treating restless leg syndrome and periodic limb movement disorder, the method comprising:
providing a treatment system comprising:
a sleeve to surround a portion of a subject;
vibration apparatus coupled to the sleeve to vibrate the sleeve;
locating the sleeve on the leg of the patient; and
vibrating the sleeve with the vibration apparatus.

10. The method of claim 9, wherein the treatment system further comprises motion sensing apparatus, and wherein the method further comprises monitoring the motion of the subject using the motion sensing apparatus to activate the vibration apparatus when the subject is moving.

11. The method of claim 9, wherein the treatment system further comprises electroencephalography apparatus, and wherein the method further comprises locating an electrode of the electroencephalography apparatus proximate a selected portion of the subject's brain.

12. The method of claim 11, wherein the selected portion of the subject's brain comprises the postcentral gyrus.

13. The method of claim 11, wherein the selected portion of the subject's brain comprises the cerebellum.

14. The method of claim 11, wherein the method further comprises monitoring the selected portion of the subject's brain with the electroencephalography apparatus to obtain neural activity data, and vibrating the sleeve with the vibration apparatus when the neural activity data indicates that the subject is moving.

15. The method of claim 9, wherein the treatment system further comprises electromyography apparatus.

16. The method of claim 15, wherein the method further comprises monitoring the selected portion of the subject's body with the electroencephalography apparatus to obtain muscular activity data, and activating the vibration apparatus when the muscular activity data indicates that the subject is moving.

17. The method of claim 9, wherein vibrating the sleeve comprises vibrating the sleeve at one or more selected frequencies.

18. The method of claim 17, wherein the one or more selected frequencies comprise a first frequency and a second frequency, wherein the first frequency is greater than the second frequency.

19. The method of claim 17, wherein the one or more selected frequencies are about 150 hertz or less.

20. The method of claim 17, wherein the one or more selected frequencies are about 100 hertz or less.

21. The method of claim 17, wherein the one or more selected frequencies are about 65 hertz or less.

22. The method of claim 9, wherein vibrating the sleeve comprises vibrating the sleeve for a selected length of time.

23. The method of claim 22, the method further comprises selecting the selected length of time.

24. The method of claim 9, wherein vibrating the sleeve comprises intermittently vibrating the sleeve.

25. The method of claim 9, wherein the sleeve surrounds at least a portion of the leg between the foot and the knee of the subject.

26. The method of claim 9, wherein the sleeve surrounds at least a portion of the leg between the foot and the thigh of the subject.

27. The method of claim 9, wherein the vibration apparatus comprises two or more vibration units spaced apart from one another on the sleeve.

28. The method of claim 9, wherein the vibration apparatus comprises a self-contained power source.
